(19) United States
(12) Patent Application Publication
(10) Pub. No.: US 2018/0296542 A1
(43) Pub. Date: Oct. 18, 2018

(54) IMINOSUGARS FOR IMPROVING BONE MINERAL DENSITY IN BONE DISEASE

(71) Applicant: Saint Louis University, St. Louis, MO (US)

(72) Inventor: Adriana M. Montaño Suarez, St. Louis, MO (US)

(21) Appl. No.: 15/762,258

(22) PCT Filed: Sep. 22, 2016
(86) PCT No.: PCT/US16/53167

§ 371 Date: Mar. 22, 2018

Related U.S. Application Data
(60) Provisional application No. 62/222,012, filed on Sep. 22, 2015.

(51) Int. Cl. A61K 31/445; A61P 19/08

(52) U.S. Cl. CPC A61K 31/445; A61P 19/08

(57) ABSTRACT

Disclosed are methods for treating bone diseases. More particularly, the present disclosure relates to methods of treating osteopenia, osteoporosis, osteomalacia, osteoarthritis, and hypophosphatasia. The present disclosure also relates to methods for improving bone mineral density.

### CROSS-REFERENCE TO RELATED APPLICATIONS

This application claims priority to U.S. Provisional Application No. 62/222,012, filed on Sep. 22, 2015, the disclosure of which is incorporated by reference in its entirety.

### STATEMENT IN SUPPORT FOR FILING A SEQUENCE LISTING

A paper copy of the Sequence Listing and a computer readable form of the Sequence Listing containing the file named “SLU15-029PCT_ST25.txt”, which is 832 bytes in size (as measured in MICROSOFT WINDOWS® EXPLORER), are provided herein and are herein incorporated by reference. This Sequence Listing consists of SEQ ID NO:1-3.

### BACKGROUND OF THE DISCLOSURE

The present disclosure relates generally to methods for treating bone diseases. More particularly, the present disclosure relates to methods of treating patients for osteopenia, osteoporosis, osteomalacia, osteoarthritis, and hypophosphatasia. The present disclosure also relates to methods for improving bone mineral density.

The human skeleton is a weight-bearing framework that protects vital organs and provides structural support for the body. The osseous tissue that forms the skeletal system can be classified according to cross-section texture as either cortical or trabecular. Cortical, or compact, bone is characterized as dense and forms the hard outer shell. Inside this shell is the trabecular, or cancellous, bone that is filled with cavities and has a more spongey texture. Trabecular bone is primarily located at the ends of the long bones in the epiphysis and metaphysis, while the diaphysis, or bone shaft, almost entirely consists of cortical bone. BMD reflects bone health. Therefore, abnormal BMD is often associated with many bone diseases. Specifically, low BMD is found in patients with osteopenia, osteoporosis, osteomalacia, osteoarthritis, and hypophosphatasia.

Hypophosphatasia (HPP) is a genetic bone disorder occurring in every 1 in 100,000 individuals. There are over 200 reported mutations in the tissue nonspecific isozyme (TNSALP) of alkaline phosphatase gene (TLPL gene) that result in the deficiency of the TNSALP enzyme and cause HPP. The four classifications of human alkaline phosphatase (ALP) are tissue-nonspecific, intestinal, placenta, and germ cell. TNSALP is the form that is abundant in various tissues, such as liver, kidney, and bone, and plays an essential role in the proper mineralization of bone tissue by preventing the buildup of its natural substrates, phosphoethanolamine (C₂H₈NO₄P [PEA]), inorganic pyrophosphate (P₂O₇⁴⁻[PPi]), and pyridoxal 5′-phosphate (vitamin B6 active form, C₈H₁₀NO₆P [PLP]), that leads to skeletal deterioration. More specifically, TNSALP hydrolyzes PPi, an inhibitor of mineralization, into inorganic phosphate (Pi), which is used to create the hydroxyapatite (a calcium phosphate) (FIG. 1). Hydroxyapatite is a main constituent of bone mineral and the bone matrix that is deposited extracellularly during bone mineralization (FIG. 2).

Categorized by age at diagnosis, HPP ranges from milder cases, such as adult, pseudohypophophatasia, and odontohypophosphatasia, to the more severe perinatal, infantile, and child forms (Table 1).

TABLE 1
Symptoms of Hypophosphatasia

| Types of Hypophosphatasia | Symptoms |
|---|---|
| Perinatal (lethal) | caput membranaceum |
| | respiratory failure |
| | short and bowed limbs |
| | inadequate bone calcification |
| Perinatal (benign)* | (same as lethal perinatal) |
| Infantile** | premature loss of deciduous teeth |
| | rickets/bowed legs |
| | respiratory complications |
| | premature craniosynostosis |
| | hypercalcemia |
| | seizures |
| Child | fractures |
| | bone pain |
| | early loss of teeth |
| | dolichocephalic skull |
| | enlarged joints |
| | inflammation |
| | rickets |
| Adult | fractures |
| | osteomalacia |
| | early loss of adult dentition |
| | pseudogout |
| Odontohypophosphatasia*** | dental manifestations (loss of teeth) |
| Pseudohypophosphatasia**** | (same as infantile) |

*skeletal deformities improve during the third trimester of pregnancy
**not present during first six months of postnatal development
***no skeletal abnormalities
****normal serum ALP activity

Generally, acute cases of HPP are autosomal recessive, whereas moderate forms are autosomal dominant. The severity of the symptoms is inversely correlated with the age at diagnosis. Perinatal HPP is often fatal due to extreme skeletal hypomineralization, resulting in inadequate calcification of bone, short and bowed limbs, respiratory failure, and caput membranaceum. Caput membranaceum refers to a soft calvarium (skullcap) due to incomplete ossification of skull that leads to craniotabes (softening and thinning of skull) and wide-open fontanelles (soft spots). The perinatal benign form of HPP has similar effects, but skeletal deformities improve during the third trimester of pregnancy. Infantile HPP may not present during the first six months of postnatal development, during which babies appear normal. The onset of the infantile disease results in premature loss of deciduous teeth, rickets/bowed legs, respiratory complications, premature craniosynostosis (sutures between skull bones fuse before the brain fully forms and prevent normal brain and skull growth), hypercalcemia (abnormally high calcium levels), and seizures. Fractures, bone pain, early loss of teeth, dolichocephalic skull (abnormally long skull), enlarged joints, inflammation, and rickets are associated with childhood HPP. Adult HPP is often characterized by pathologic fractures as well as osteomalacia (softening of bones), early loss of adult dentition, and pseudogout (form of arthritis resulting in sudden, painful, swelling of joints). Odontophosphatasia is limited to dental manifestations, primarily the loss of teeth, and does not result in skeletal abnormalities. Pseudohypophophatasia presents similarly to infantile HPP but serum ALP activity is normal.

Currently, the only established and FDA-approved medical therapy for HPP is enzyme replacement therapy (ERT). ERT studies in Akp2^{-/-} mice administered untagged, native human TNSALP exhibited longer lives and demonstrated better growth and normal fertility as compared to untreated knock-out animals. After 6 months, however, hypomineralization and abnormal proliferative chondrocytes presented in the mice. Akp2^{-/-} mice injected with a bone-targeted, recombinant form of human TNSALP (sALP-FcD₁₀) did not exhibit signs of epilepsy and lacked skeletal defects and dental manifestations and also had normal substrate concentrations. Patients treated by ERT with the bone-targeted, recombinant human TNSALP ENB-0040 (asfotase alfa) showed radiographic improvements in mineralization, normal substrate levels, and better pulmonary and physical function. Although ERT has had success in treating patients with HPP, the costs of this procedure limit its accessibility. Accordingly, there exists a need to develop alternative methods for treating bone diseases.

### SUMMARY OF THE DISCLOSURE

The present disclosure relates generally to methods for treating bone diseases. More particularly, the present disclosure relates to methods of treating osteopenia, osteoporosis, osteomalacia, osteoarthritis, and hypophosphatasia. The present disclosure also relates to methods for increasing bone mineral density.

In one aspect, the present disclosure is directed to a method for treating osteopenia. The method comprises administering a composition comprising an iminosugar.

In one aspect, the present disclosure is directed to a method for treating osteoporosis. The method comprises administering a composition comprising an iminosugar.

In one aspect, the present disclosure is directed to a method for treating osteomalacia. The method comprises administering a composition comprising an iminosugar.

In one aspect, the present disclosure is directed to a method for treating osteoarthritis. The method comprises administering a composition comprising an iminosugar.

In one aspect, the present disclosure is directed to a method for treating hypophosphatasia. The method comprises administering a composition comprising an iminosugar.

In one aspect, the present disclosure is directed to a method for increasing bone mineral density. The method comprises administering a composition comprising an iminosugar.

### DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the hydrolysis of inorganic pyrophosphate (PPi) to inorganic phosphate (Pi) by alkaline phosphatase (ALP). PPi is a natural substrate of ALP and an inhibitor of mineralization. ALP serves as a catalyst in the reaction that hydrolyzes PPi to Pi. Pi is required to make hydroxyapatite, which is deposited in the extracellular matrix during bone mineralization.

FIG. 2 depicts the mechanism of bone mineralization. ALP is crucial for the proper mineralization of bone. ALP hydrolyzes its natural substrate PPi to Pi for the creation of hydroxyapatite. Hydroxyapatite is necessary for the mineralization as this calcium phosphate deposited extracellularly during the process.

FIG. 3 is a graph depicting average BMDs of all treatment groups. BMD increased with Miglustat dosage across all genotypes, with the exception of wild-type mice treated with 1200 mg/kg of Miglustat. Standard error bars are not included for homozygous treatment groups because the counts were not sufficient for statistical analysis.

FIGS. 4A-4T are depict phenotype (FIGS. 4A-4D), X-Ray images of ventral (FIGS. 4E-4H), X-Ray images of side (FIGS. 4I-4L), X-Ray images of femur (FIGS. 4M-4P)), and microCT scans (FIGS. 4Q-4T) of wild-type mice treated with PBS. This group served as a control because ALP activity levels were normal in wild-type mice and no Miglustat treatment was adminstered.

FIGS. 5A-5Y depict phenotype (FIGS. 5A-5E), X-Ray images of ventral (FIGS. 5F-5J), X-Ray images of side (FIGS. 5K-50), X-Ray images of femur (FIGS. 5P-5T)), and microCT scans (FIGS. 5U-5Y) of wild-type mice treated with Miglustat (400 mg/kg). This treatment group had the highest average BMD and therefore most bone mineralization.

FIGS. 6A-6U depict phenotype (FIGS. 6A-6D), X-Ray images of ventral (FIGS. 6E-6H), X-Ray images of side (FIGS. 6I-6L), X-Ray images of femur (FIGS. 6M-6P), and microCT scans (FIGS. 6Q-6U) of wild-type mice treated with Miglustat (1200 mg/kg).

FIGS. 7A-7T depict phenotype (FIGS. 7A-7D), X-Ray images of ventral (FIGS. 7E-7H), X-Ray images of side (FIGS. 7I-7L), X-Ray images of femur (FIGS. 7M-7P), and microCT scans (FIGS. 7Q-7T) of heterozygous mice treated with PBS. Heterozygotes exhibited roughly 50% of wild-type serum ALP activity levels. This treatment group had a lower average BMD than the wild-types on PBS and a higher average BMD than the knockouts on PBS.

FIGS. 8A-8O depict phenotype (FIGS. 8A-8C), X-Ray images of ventral (FIGS. 8D-8F), X-Ray images of side (FIGS. 8G-8I), X-Ray images of femur (FIGS. 8J-8L), and microCT scans (FIGS. 8M-8O) of heterozygous mice treated with Miglustat (400 mg/kg). This treatment group had a higher average BMD value than the PBS heterozygotes, and therefore better bone mineralization due to Miglustat.

FIG. 9A-9T depict phenotype (FIGS. 9A-9D), X-Ray images of ventral (FIGS. 9E-9H), X-Ray images of side (FIGS. 9I-9L), X-Ray images of femur (FIGS. 9M-9P), and microCT scans (FIGS. 9Q-9T) of heterozygous mice treated with Miglustat (1200 mg/kg). This treatment group had an average BMD that matched and surpassed that of the wild-types on PBS, and therefore reached the desired BMD through Miglustat treatment.

FIGS. 10A-10J depict phenotype (FIGS. 10A and 10B), X-Ray images of ventral (FIGS. 10C and 10D), X-Ray images of side (FIGS. 10E and 10F), X-Ray images of femur (FIGS. 10G and 10H), and microCT scans (FIGS. 10I and 10J) of homozygous mice treated with PBS. This group served as a mouse model of infantile HPP because ALP activity is roughly 1% of wild-type levels and no Miglustat treatment was given. MicroCT scans showed clear evidence of improper cortical bone mineralization at the diaphysis. This treatment group had the lowest average BMD and least bone mineralization.

FIGS. 11A-11J depict phenotype (FIGS. 11A and 11B), X-Ray images of ventral (FIGS. 11C and 11D), X-Ray images of side (FIGS. 11E and 11F), X-Ray images of femur (FIGS. 11G and 11H), and microCT scans (FIGS. 11I and 11J) of homozygous mice treated with Miglustat (400 mg/kg). MicroCT scans showed clear evidence of improper cortical bone mineralization at the diaphysis. However, this treatment group had a higher average BMD and therefore better mineralization than the knockouts on PBS.

FIG. 12 is a graph depicting impeded growth in mice administered high doses of Miglustat.

FIGS. 13A-13I are X-Rays of femurs from Wild-type mice administered PBS (WT-PBS; FIG. 13A), Heterozygous mice administered PBS (Het-PBS; FIG. 13B), Homozygous mice administered PBS (KO-PBS; FIG. 13C), Wild-type mice administered 400 mg/kg Miglustat (WT-400 mg/kg; FIG. 13D), Heterozygous mice administered 400 mg/kg Miglustat (Het-400 mg/kg; FIG. 13E), Homozygous mice administered 400 mg/kg Miglustat (KO-400 mg/kg; FIG. 13F), Wild-type mice administered 1200 mg/kg Miglustat (WT-1200 mg/kg; FIG. 13G), Heterozygous mice administered 400 mg/kg Miglustat (Het-1200 mg/kg; FIG. 13H), Homozygous mice administered 400 mg/kg Miglustat (KO-1200 mg/kg; FIG. 13I) showing increased bone at primary ossification centers (arrowheads).

FIG. 14 is a graph depicting cortical thickness (Ct.Th, μm) in Wild-type (WT), Heterozygotes (Het) and Homozygotes (KO) administered PBS, 400 mg/kg, and 1200 mg/kg Miglustat.

FIG. 15 is a graph depicting total cross-sectional area (Tt.Ar, mm²) in Wild-type (WT), Heterozygotes (Het) and Homozygotes (KO) administered PBS, 400 mg/kg, and 1200 mg/kg Miglustat.

FIG. 16 is a graph depicting the cortical bone area (Ct.Ar, mm²) in Wild-type (WT), Heterozygotes (Het) and Homozygotes (KO) administered PBS, 400 mg/kg, and 1200 mg/kg Miglustat.

FIG. 17 is a graph depicting the normalized bone area—cortical bone volume fraction in Wild-type (WT), Heterozygotes (Het) and Homozygotes (KO) administered PBS, 400 mg/kg, and 1200 mg/kg Miglustat.

FIG. 18 is a graph depicting tissue mineral density (mg HA/cm³) in Wild-type (WT), Heterozygotes (Het) and Homozygotes (KO) administered PBS, 400 mg/kg, and 1200 mg/kg Miglustat.

FIGS. 19A-19C are microCT images of trabecular bone in Wild-type mice (WT-PBS), Heterozygotes (Het-PBS), and Homozygotes (KO-PBS) treated with PBS showing visible different trabeculae in Akp^{-/-} (KO) mice.

FIGS. 20A-20D are microCT images of trabecular bone in Wild-type mice treated with PBS (WT-PBS, FIG. 20A) and Homozygotes treated with PBS (KO-PBS, FIG. 20B), Homozygotes treated with 400 mg/kg Miglustat (KO-400 mg/kg, FIG. 20C), and Homozygotes treated with 1200 mg/kg Miglustat (KO-1200 mg/kg, FIG. 20D).

FIG. 21 is a graph depicting bone volume fraction in Wild-type (WT), Heterozygotes (Het) and Homozygotes (KO) administered PBS, 400 mg/kg, and 1200 mg/kg Miglustat.

FIG. 22 is a graph depicting trabecular thickness (Tb.Th, mm) in Wild-type (WT), Heterozygotes (Het) and Homozygotes (KO) administered PBS, 400 mg/kg, and 1200 mg/kg Miglustat.

FIG. 23 is a schematic illustrating how the trabecular thickness measurement was taken (as disclosed in Bouxsein et al., J. Bone Miner Res. 2010 July; 25(7):1468-86).

FIG. 24 is a graph depicting trabecular separation (Tb.Sp, mm) in Wild-type (WT), Heterozygotes (Het) and Homozygotes (KO) administered PBS, 400 mg/kg, and 1200 mg/kg Miglustat.

FIG. 25 is a schematic illustrating how the trabecular thickness measurement was taken (as disclosed in Bouxsein et al., J. Bone Miner Res. 2010 July; 25(7):1468-86).

FIG. 26 is a graph depicting trabecular number (Tb.N, 1/mm) in Wild-type (WT), Heterozygotes (Het) and Homozygotes (KO) administered PBS, 400 mg/kg, and 1200 mg/kg Miglustat.

FIG. 27 is a graph depicting structure model index (SMI) in Wild-type (WT), Heterozygotes (Het) and Homozygotes (KO) administered PBS, 400 mg/kg, and 1200 mg/kg Miglustat.

FIG. 28 is a graph depicting connectivity density (Conn. D, 1/mm³) in Wild-type (WT), Heterozygotes (Het) and Homozygotes (KO) administered PBS, 400 mg/kg, and 1200 mg/kg Miglustat.

FIG. 29 is a graph depicting bone volume fraction of lumbar vertebrae in Wild-type (WT) and Homozygotes (KO) administered PBS, 400 mg/kg, and 1200 mg/kg Miglustat. IS THIS FOR VERTEBRA?

FIG. 30 is a graph depicting trabecular thickness (Tb.Th, mm) of lumbar vertebrae in Wild-type (WT) and Homozygotes (KO) administered PBS, 400 mg/kg, and 1200 mg/kg Miglustat.

FIG. 31 is a graph depicting trabecular separation (Tb.Sp, mm) of lumbar vertebrae in Wild-type (WT) and Homozygotes (KO) administered PBS, 400 mg/kg, and 1200 mg/kg Miglustat.

FIG. 32 is a graph depicting trabecular number (Tb.N, 1/mm) of lumbar vertebrae in Wild-type (WT) and Homozygotes (KO) administered PBS, 400 mg/kg, and 1200 mg/kg Miglustat.

FIG. 33 is a graph depicting structure model index (SMI) of lumbar vertebrae in Wild-type (WT) and Homozygotes (KO) administered PBS, 400 mg/kg, and 1200 mg/kg Miglustat.

FIG. 34 is a graph depicting connectivity density (Conn. D, 1/mm³) of lumbar vertebrae in Wild-type (WT) and Homozygotes (KO) administered PBS, 400 mg/kg, and 1200 mg/kg Miglustat.

FIG. 35 is a graph depicting degree of anisotropy (DA) of lumbar vertebrae in Wild-type (WT) and Homozygotes (KO) administered PBS, 400 mg/kg, and 1200 mg/kg Miglustat.

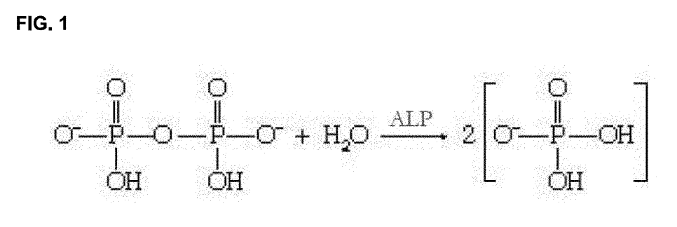

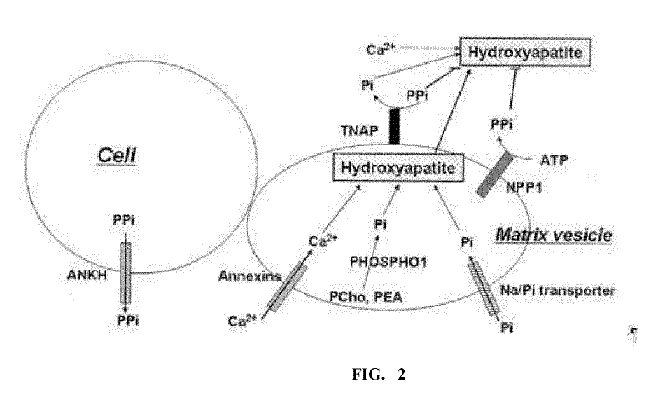

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

In accordance with the present disclosure, methods have been discovered for treating bone diseases. In particular, methods have been discovered for treating osteopenia, osteoporosis, osteomalacia, osteoarthritis, and hypophosphatasia. Methods have also been discovered for increasing bone mineral density.

As used herein, “a subject in need thereof” refers to a subject having, susceptible to or at risk of having a specified disease, disorder, or condition. More particularly, in the present disclosure, the methods of treating bone diseases and increasing bone mineral density can be used with a subset of subjects who have, are susceptible to or at elevated risk for experiencing osteopenia, osteoporosis, osteomalacia, osteoarthritis, and hypophosphatasia, and/or subjects who have, are susceptible to or at elevated risk for low bone mineral density. Subjects may be susceptible to or at elevated risk for osteopenia, osteoporosis, osteomalacia, osteoarthritis, and hypophosphatasia and/or low bone mineral density due to family history, age, environment, clinical presentation, and/or lifestyle. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need thereof” the specific conditions noted herein), not all individuals will fall within the subset or subclass of individuals as described herein.

As used herein, “increasing” bone mineral density refers to an increase in the measure of bone mineral density as compared to a bone mineral density measurement taken prior to administration of treatment. Bone mineral density can be determined by methods known to those skilled in the art such as, for example, Dual-energy X-ray absorptiometry (DXA), Peripheral dual-energy X-ray absorptiometry (P-DXA), Dual photon absorptiometry (DPA), ultrasound, MicroCT scanning, and combinations thereof.

In one aspect, the present disclosure is directed to a method for treating osteopenia. The method comprises administering a composition comprising an iminosugar. Suitable immunosugars include multivalent iminosugars (as provided in Laigre et al., Carbohydrate Research 429 (2016):98-104), sp2 iminosugars (as provided in Mena-Barragan et al., European J. of Medicinal Chemistry 121 (2016): 880-891), and iminosugar based galactosides. Other suitable iminosugars include miglustat, eligustat, Bicyclic 1-Deoxygalactonojirimycin derivative (as provided in Takai et al., Molecular Therapy 21(3) (2013):526-532) and combinations thereof.

In one aspect, the present disclosure is directed to a method for treating osteoporosis. The method comprises administering a composition comprising an iminosugar. Suitable iminosugars are described herein.

In one aspect, the present disclosure is directed to a method for treating osteomalacia. The method comprises administering a composition comprising an iminosugar. Suitable iminosugars are described herein.

In one aspect, the present disclosure is directed to a method for treating osteoarthritis. The method comprises administering a composition comprising an iminosugar. Suitable iminosugars are described herein.

In one aspect, the present disclosure is directed to a method for treating hypophosphatasia. The method comprises administering a composition comprising an iminosugar. Suitable iminosugars are described herein.

In one aspect, the present disclosure is directed to a method for increasing bone mineral density. The method comprises administering a composition comprising an iminosugar. Suitable iminosugars are described herein.

Iminosugars can be in a form of a salt derived from an inorganic or organic acid. Pharmaceutically acceptable salts and methods for preparing salt forms are disclosed, for example, in Berge et al. (J. Pharm. Sci. 66:1-18, 1977). Examples of appropriate salts include, for example, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

Miglustat ((2R,3R,4R,5S)-1-butyl-2-(hydroxymethyl)piperidine-3,4,5-triol) is an iminosugar (N-butyldeoxynojirimycin) known for inhibiting glucosylceramide synthase biosynthesis and used for treating the lysosomal storage disorder Gaucher's disease (GD). A study on patients with Type 1 GD found that Miglustat treatment increased BMD and alleviated bone pain. However, the complete mechanism of Miglustat is still unknown.

Suitable dosages of miglustat can range from about 1 mg/kg/day to about 1,200 mg/kg based on an average human weight of about 70 kg to about 85 kg, including from about 3 mg/kg/day to about 400 mg/kg/day.

Eligustat (N-[(1R,2R)-1-(2,3-Dihydro-1,4-benzodioxin-6-yl)-1-hydroxy-3-(1-pyrrolidinyl)-2-propanyl]octanamide) is known for inhibiting glucosylceramide synthase and is used as a treatment for Gaucher's disease.

Compositions can further include a pharmaceutically acceptable carrier and/or a component useful for delivering the composition to a subject. Numerous pharmaceutically acceptable carriers useful for delivering the compositions to a human and components useful for delivering the composition to other animals, such as cattle are known in the art. Addition of such carriers and components is within the level of ordinary skill in the art.

The amount of the iminosugar that should be administered to a cell or animal can depend upon numerous factors well understood by one of skill in the art, such as the molecular weight of the agent and the route of administration. Actual dosage levels of iminosugar (and other active agents) in the pharmaceutical compositions can vary so as to administer an amount of the active compound(s) that is effective to achieve the desired therapeutic response for a particular patient. The selected dose level can depend on the activity of the agent, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose can be divided into multiple doses for purposes of administration, for example, two to four doses per day. It should be understood, however, that the specific dose level for any particular patient can depend on a variety of factors, including the body weight, general health, diet, time and route of administration and combination with other therapeutic agents and the severity of the condition or disease being treated.

Compositions can be administered orally, topically, parenterally, systemically, by a pulmonary route, and combinations thereof. As used herein, the term “parenteral” includes subcutaneous, intravenous, intraarterial, intrathecal, and injection and infusion techniques. The iminosugar containing compositions are preferentially administered intraperitoneally. Compositions can be administered systemically in oral solid formulations, ophthalmic, suppository, aerosol, topical and other similar formulations. For example, the physical form of the composition can be in the form of a powder, tablet, capsule, lozenge, gel, solution, suspension, syrup, and the like. In addition to the active agent, such compositions can contain pharmaceutically-acceptable carriers and other ingredients known to enhance and facilitate drug administration. Other possible formulations, such as nanoparticles, liposomes resealed erythrocytes, and immunologically based systems may also be used to administer the agent.

### EXAMPLES

### Example 1

TNSALP-null (Akp2^{-/-}) mice served as an in vivo model of infantile HPP to analyze the effects of Miglustat as a treatment for HPP through BMD measurements. In comparison to wild-type serum levels, Akp2^{-/-} mice exhibit ˜1% of ALP activity, while heterozygous mice display roughly 50%. Resembling patients with HPP, elevated substrate concentrations of PEA, PPi, and PLP were found in urine samples from knock-out mice. Knockouts did not radiographically show skeletal defects at birth, but later developed the disease, similar to patients with infantile HPP.

The eight treatment groups included three genotypes (wild-type, heterozygous, and homozygous) and three treatments (Phosphate-buffered saline (PBS), Miglustat (400 mg/kg), Miglustat (1200 mg/kg). After one month of treatment, ventral and side x-rays were taken and the femur was microCT scanned to determine BMD. Higher Miglustat doses led to higher average BMD values, with the exception of one treatment group, indicating that Miglustat could be used to increase BMD and to treat bone diseases including HPP.

### Mouse Model

Heterozygous mice were purchased from Jackson Laboratory (Bar Harbor, Me.) and bred for homozygous knock-outs that phenocopy infantile HPP. All experiments were conducted with the highest standards of humane animal care at Saint Louis University under an approved IACUC protocol.

### Genotyping

Mice were genotyped as wild-type, heterozygous, or homozygous through DNA extraction, polymerase chain reaction (PCR), and gel electrophoresis. After DNA was extracted from an ear tissue sample, PCR was used to generate copies of the DNA for gel electrophoresis (2% agarose and Tris-Acetate-EDTA (TAE) buffer). The following primers were used: 5′-AGG GGG ATG TGC TGC AAG GCG ATT-3′ (SEQ ID NO:1), 5′-CTG GCA CAA AAG AGT TGG TAA GGC-3′ (SEQ ID NO:2), 5′-GAT CGG AAC GTC AAT TAA CGT CAA-3′ (SEQ ID NO:3, wild-type allele: 160 bp, mutant allele: 195 bp).

### Treatment

At the age of one month, mice were administered 100 μL injections of phosphate-buffered saline (PBS) with 0 mg/kg, 400 mg/kg, or 1200 mg/kg of Miglustat (250 mg/mL) to the peritoneal cavity for gradual diffusion of the drug. Mice were massed each day to adjust Miglustat dosages to body weight. Intraperitoneal injections (IP) continued daily for one month before mice were euthanized with CO₂ at the age of two months. Mice were on a Pyridoxine Supplement Diet (“Vitamin B6 Diet”) to prevent seizures. NAPA NECTAR™ (Systems Engineering™) was provided every alternating day to prevent dehydration. Urine samples were collected once a week and blood samples were collected from cheek tissue at the beginning and end of treatment.

### Scanning

Full-body ventral and side-view x-rays were taken after euthanizing the mice. The left anatomical legs were dissected to obtain the femoral bones. Femurs were then frozen before being placed in 10% formalin the day prior to microCT scanning. Four bones were inserted into a gel cast (2.5% agarose and TAE buffer) at a time and covered with 2.5% agarose and TAE buffer to create gel capsules that were placed in sample tubes (diameter: 11.5 mm, height: 75 mm) for microCT scanning Cortical bone scans (voxel size: 12 μm) were performed at the diaphysis (femoral shaft) using a SCANCO μCT 35 system (SCANCO Medical; Brüttiselien, Switzerland).

### Analysis

MicroCT scans were analyzed using SCANCO Medical microCT software to produce three dimensional reconstructions of the bones and determine bone mineral density (BMD) as the mean/density [mg HA/ccm] of total volume (TV) (Apparent). The total length of each bone was measured as the distance from the greater trochanter to the distal end. Only the middle 20% of each femur was accounted for during cortical bone analysis.

Statistical analysis included calculating the average mean/density [mg HA/ccm] of TV (Apparent) and standard error for each treatment group to determine the estimated standard deviation of the statistics. Standard deviation and standard error were not calculated for homozygous mice due to count values below three.

### Results

A total of 29 mice (wild-type: 14, heterozygous: 11, homozygous: 4) were analyzed in this study (summarized by mouse number in Table 2). Of the 13 wild-types, four received PBS, five were injected with 400 mg/kg of Miglustat, and four were treated with 1200 mg/kg of Miglustat. Four of the 11 heterozygous mice were only treated with PBS, while three received 400 mg/kg of Miglustat, and four received 1200 mg/kg of Miglustat. Only four total homozygous mice were part of this study. Two were injected with PBS and two were treated with 400 mg/kg Miglustat. No knockouts were part of a Miglustat (1200 mg/kg) treatment group.

TABLE 2
Mouse Number, Genotype and Treatment.

| | Genotype | | |
|---|---|---|---|
| Treatment | Wild-Type | Heterozygous | Homozygous |
| PBS | 3331 | 3332 | 3333 |
| | 3342 | 3334 | 3371 |
| | 3343 | 3345 | |
| | 3344 | 3346 | |
| Miglustat (400 mg/kg) | 3316 | 3357 | 3315 |
| | 3347 | 3361 | 3329 |
| | 3358 | 3362 | |
| | 3359 | | |
| | 3363 | | |
| Miglustat (1200 mg/kg) | 3368 | 3317 | |
| | 3369 | 3374 | |
| | 3378 | 3375 | |
| | 3380 | 3370 | |

Full-body ventral and side x-rays were taken and microCT scans were performed on the femurs of each mouse. The bone mineral densities of the femurs were recorded in units of hydroxyapatite density as the mean/density [mg HA/ccm] of TV (apparent). The average bone mineral densities (average mean/density [mg HA/ccm] of TV (apparent) were then calculated for each treatment group. Statistical analysis, including standard deviation and standard error, was only performed for the wild-types and heterozygotes due to insufficient data for the homozygous mice. A minimum of three mice were required for statistical analysis.

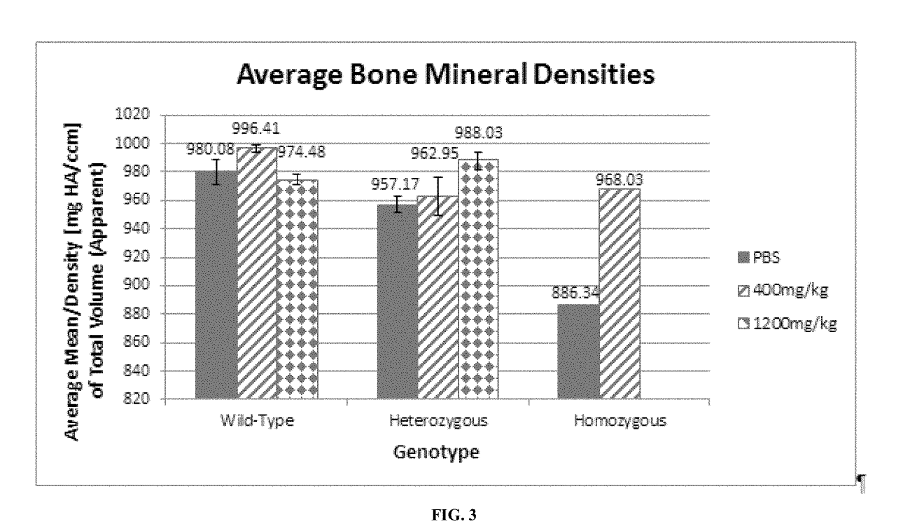

Of the mice only injected with PBS, the wild-types had the highest average BMD (980.08±8.58 mg HA/ccm, n=4) (Tables 3 and 4), followed by the heterozygotes (957.17±5.32 mg HA/ccm, n=4), and then the homozygous knockouts (886.34 mg HA/ccm, n=2) (Tables 4, 10, & 16). Only two treatment groups (wild-type, 400 mg/kg and heterozygous, 1200 mg/kg) had average BMD values greater than that of the control wild-types receiving PBS (FIG. 3). The wild-type mice administered 400 mg/kg of Miglustat had the highest average BMD of all eight treatment groups (Table 19 & FIG. 3).

TABLE 3
BDM of Wild-Type Mice Treated with PBS.

| Mouse Number | Mean/Density [mg HA/ccm] of TV (Apparent) |
|---|---|
| 3331 | 955.45 |
| 3342 | 994.24 |
| 3343 | 988.57 |
| 3344 | 982.07 |

TABLE 4
Statistical Analysis of Wild-Type Mice Treated with PBS.

| | |
|---|---|
| Average Mean/Density [mg HA/ccm] of TV | 980.08 |
| (Apparent) | |
| Standard Deviation | 17.16 |
| Count | 4 |
| Standard Error | 8.58 |

TABLE 5
BDM of Wild-Type Mice Treated with Miglustat (400 mg/kg).

| Mouse Number | Mean/Density [mg HA/ccm] of TV (Apparent) |
|---|---|
| 3316 | 1000.00 |
| 3347 | 990.58 |
| 3358 | 991.31 |
| 3359 | 1004.30 |
| 3363 | 995.88 |

TABLE 6
Statistical Analysis of Wild-Type Mice
Treated with Miglustat (400 mg/kg).

| | |
|---|---|
| Average Mean/Density [mg HA/ccm] of TV | 996.41 |
| (Apparent) | |
| Standard Deviation | 5.82 |
| Count | 5 |
| Standard Error | 2.60 |

TABLE 7
BDM of Wild-Type Mice Treated with Miglustat (1200 mg/kg).

| Mouse Number | Mean/Density [mg HA/ccm] of TV (Apparent) |
|---|---|
| 3368 | 973.20 |
| 3369 | 985.09 |
| 3378 | 969.63 |
| 3380 | 967.00 |

TABLE 8
Statistical Analysis of Wild-Type Mice
Treated with Miglustat (1200 mg/kg).

| | |
|---|---|
| Average Mean/Density [mg HA/ccm] of TV | 974.48 |
| (Apparent) | |
| Standard Deviation | 7.25 |
| Count | 4 |
| Standard Error | 3.63 |

TABLE 9
BDM of Heterozygous Mice Treated with PBS.

| Mouse Number | Mean/Density [mg HA/ccm] of TV (Apparent) |
|---|---|
| 3332 | 970.82 |
| 3334 | 948.68 |
| 3345 | 960.39 |
| 3346 | 948.77 |

TABLE 10
Statistical Analysis of Heterozygous Mice Treated with PBS.

| | |
|---|---|
| Average Mean/Density [mg HA/ccm] of TV | 957.17 |
| (Apparent) | |
| Standard Deviation | 10.63 |
| Count | 4 |
| Standard Error | 5.32 |

TABLE 11
BDM of Heterozygous Mice Treated with Miglustat (400 mg/kg).

| Mouse Number | Mean/Density [mg HA/ccm] of TV (Apparent) |
|---|---|
| 3357 | 983.17 |
| 3361 | 937.07 |
| 3362 | 968.62 |

TABLE 12
Statistical Analysis of Heterozygous Mice
Treated with Miglustat (400 mg/kg).

| | |
|---|---|
| Average Mean/Density [mg HA/ccm] of TV | 962.95 |
| (Apparent) | |
| Standard Deviation | 23.57 |
| Count | 3 |
| Standard Error | 13.61 |

TABLE 13
BDM of Heterozygous Mice Treated with Miglustat (1200 mg/kg).

| Mouse Number | Mean/Density [mg HA/ccm] of TV (Apparent) |
|---|---|
| 3317 | 980.88 |
| 3370 | 984.82 |
| 3374 | 979.78 |
| 3375 | 1006.65 |

TABLE 14
Statistical Analysis of Heterozygous Mice
Treated with Miglustat (1200 mg/kg)

| | |
|---|---|
| Average Mean/Density [mg HA/ccm] of TV | 988.03 |
| (Apparent) | |
| Standard Deviation | 12.60 |
| Count | 4 |
| Standard Error | 6.30 |

TABLE 15
Individual BMDs of Homozygous Mice Treated with PBS.

| Mouse Number | Mean/Density [mg HA/ccm] of TV (Apparent) |
|---|---|
| 3333 | 871.02 |
| 3371 | 901.67 |

TABLE 16
Average BMD of Homozygous Mice Treated with PBS.

| | |
|---|---|
| Average Mean/Density [mg HA/ccm] of TV | 886.34 |
| (Apparent) | |
| Count | 2 |

TABLE 17
Individual BMD of Homozygous Mice
Treated with Miglustat (400 mg/kg).

| Mouse Number | Mean/Density [mg HA/ccm] of TV (Apparent) |
|---|---|
| 3315 | 881.08 |
| 3329 | 1054.98 |

TABLE 18
Average BMD of Homozygous Mice Treated
with Miglustat (400 mg/kg).

| | |
|---|---|
| Average Mean/Density [mg HA/ccm] of TV | 968.03 |
| (Apparent) | |
| Count | 2 |

TABLE 19
Average Bone Mineral Densities (mg HA/ccm).

| | Genotype | | |
|---|---|---|---|
| Treatment | Wild-Type | Heterozygous | Homozygous |
| PBS | 980.08 | 957.17 | 886.34 |
| Miglustat (400 mg/kg) | 996.41 | 962.95 | 968.03 |
| Miglustat (1200 mg/kg) | 974.48 | 988.03 | |

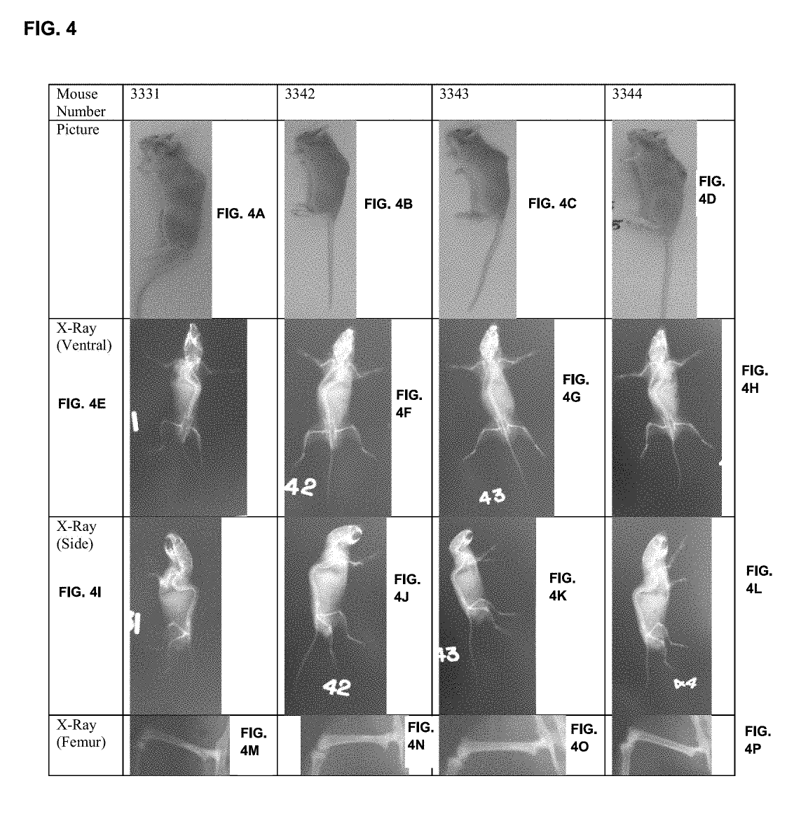

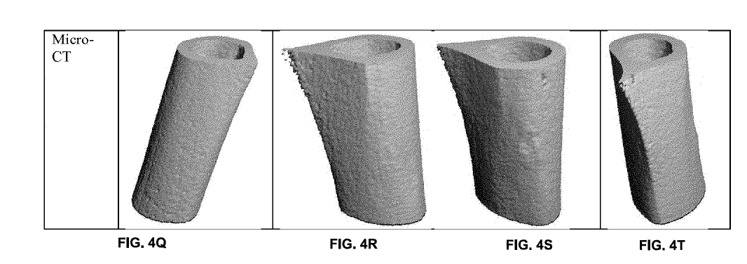

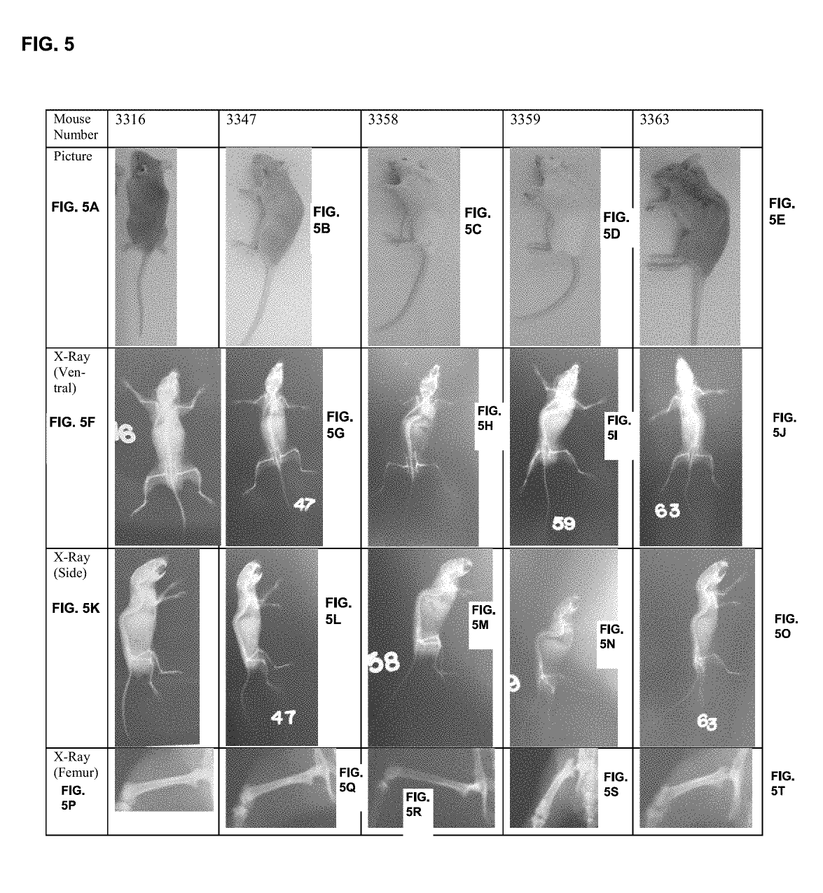

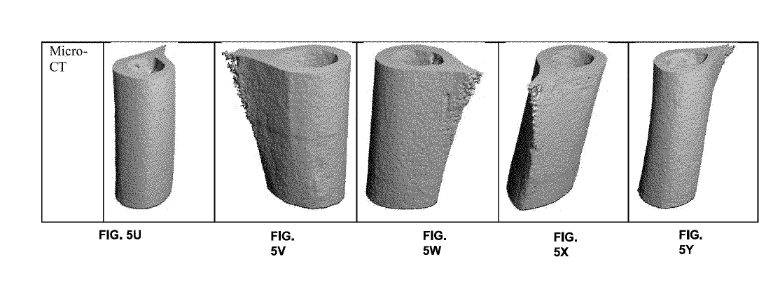

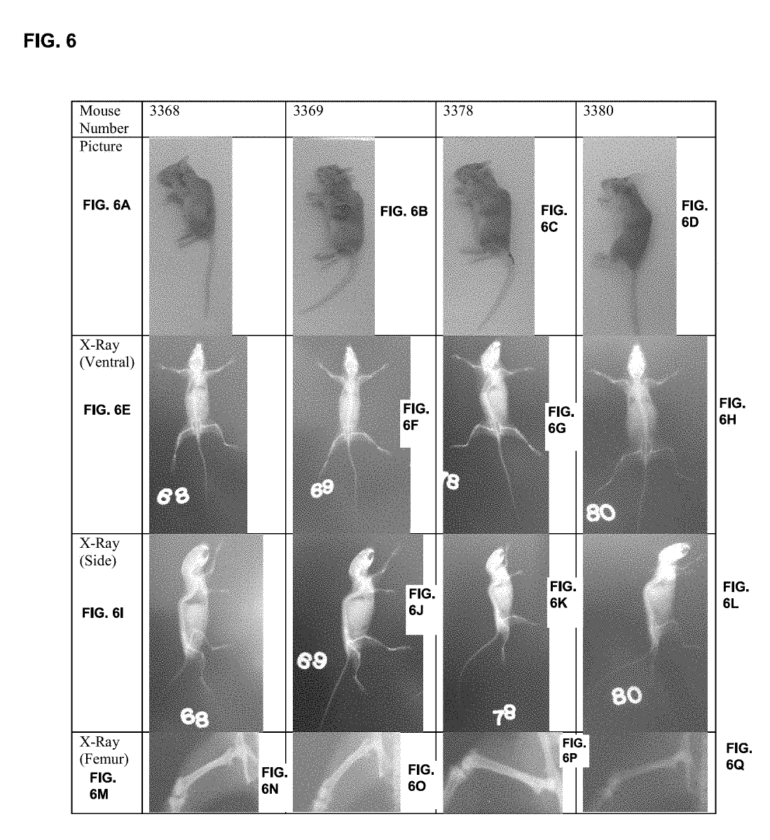

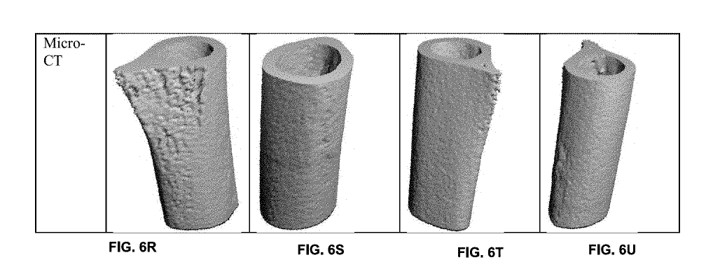

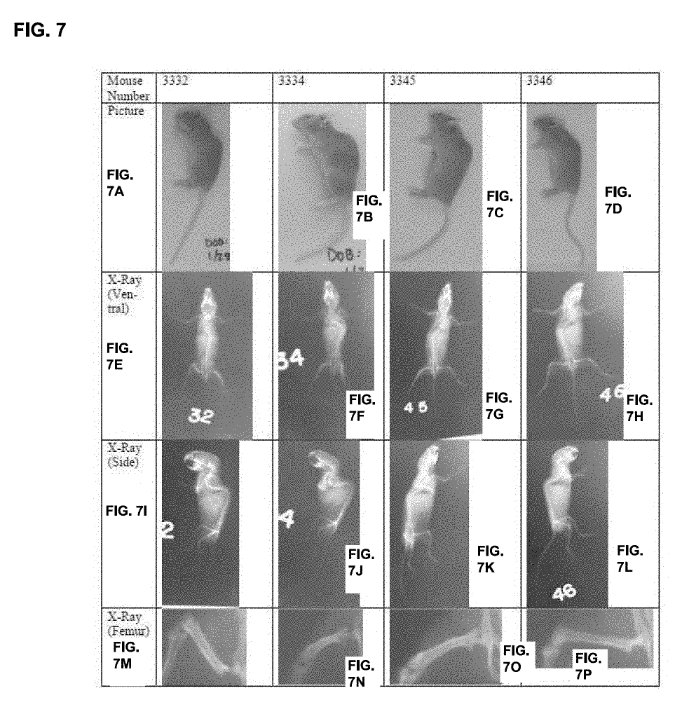

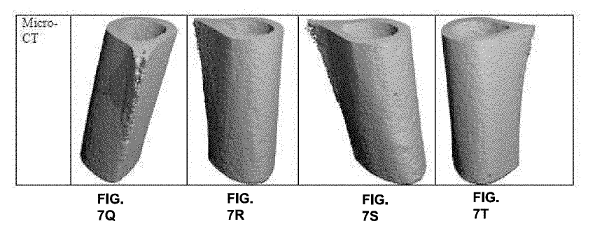

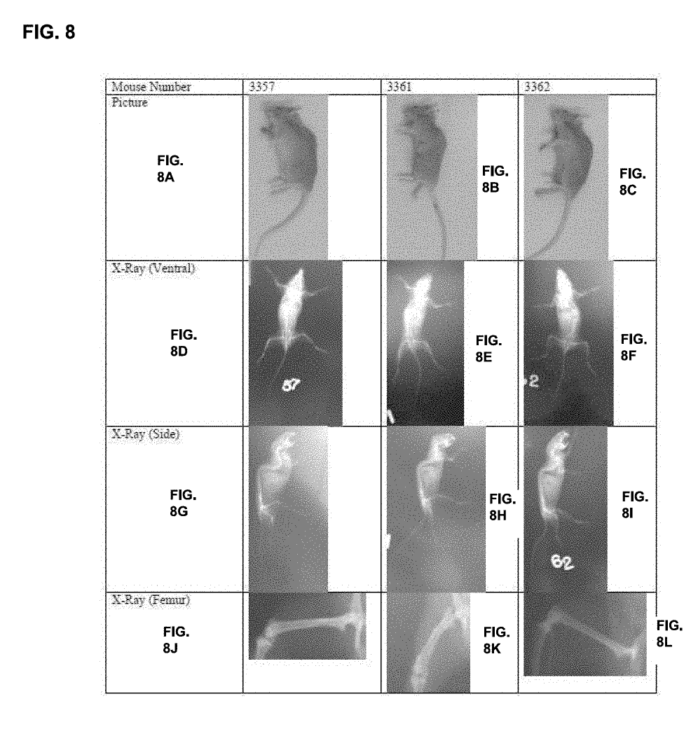

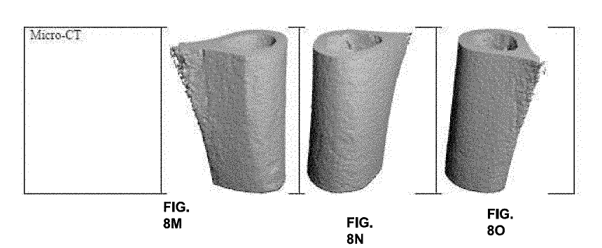

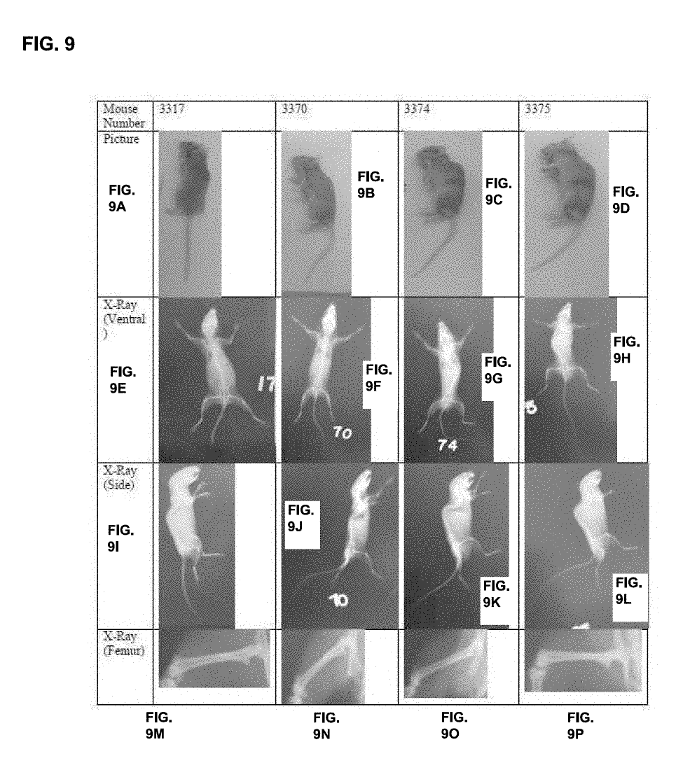

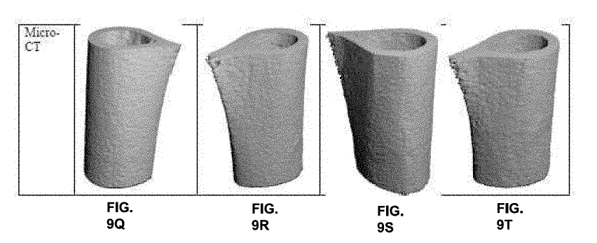

Higher Miglustat doses were associated with higher average BMD for heterozygous and homozygous mice. BMD values were collected for all three treatments in heterozygotes. The heterozygous mice administered 1200 mg/kg of Miglustat had greater mineralization of cortical bone tissue in comparison to the two other heterozygous treatment groups (FIG. 3). PBS-treated mice had the lowest average mean/density of TV (apparent) for heterozygotes (957.17±5.32 mg HA/ccm, n=4), while the 400 mg/kg Miglustat group showed a slight improvement in average BMD (962.95±13.61 mg HA/ccm, n=3), and mice administered 1200 mg/kg of Miglustat showed even greater improvement (988.03±6.30 mg HA/ccm, n=4) (Tables 10, 12, & 14). Clear differences in mineralization were not discernable in the heterozygous microCT scans between treatments (FIGS. 7-9) and in comparison to the control PBS wild-types (FIG. 4).

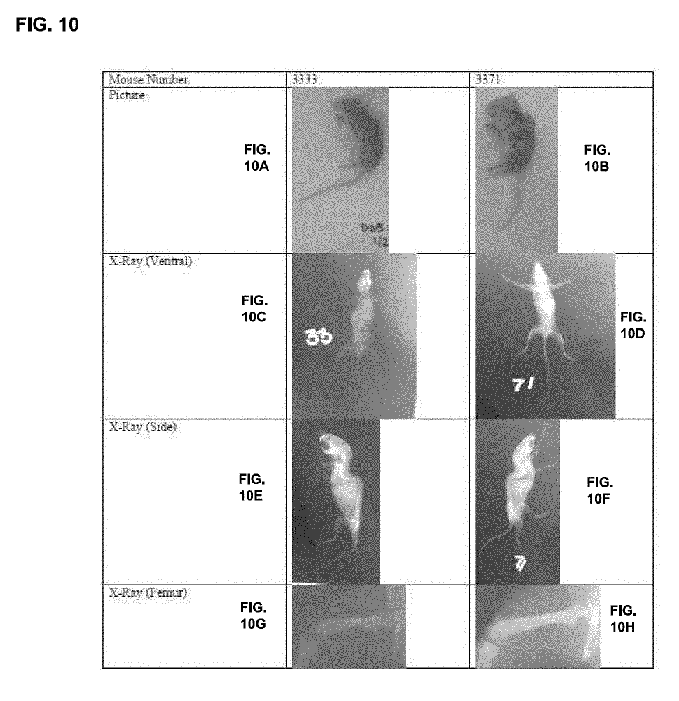

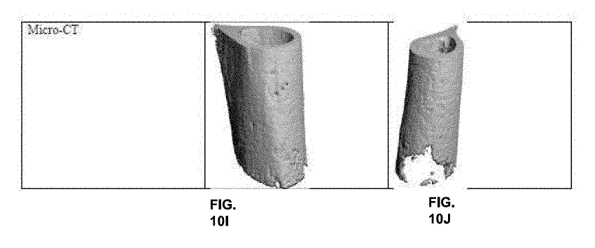

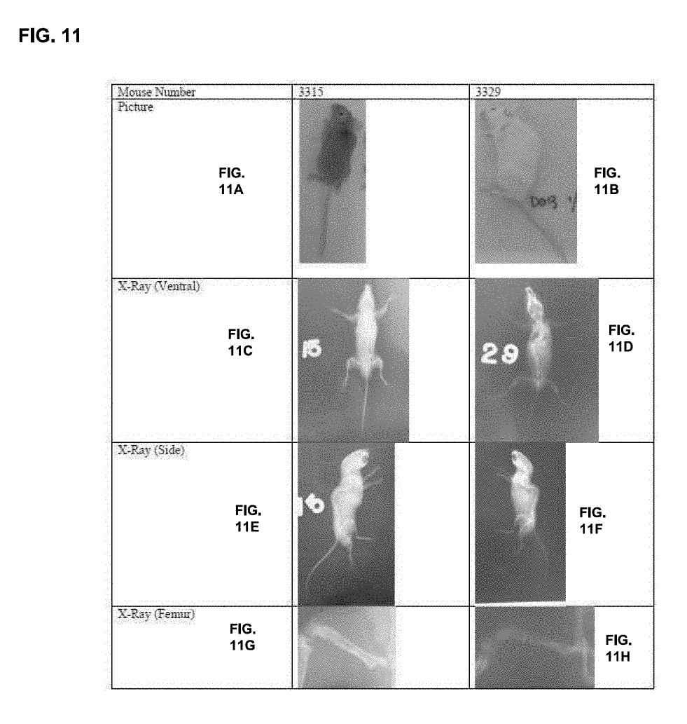

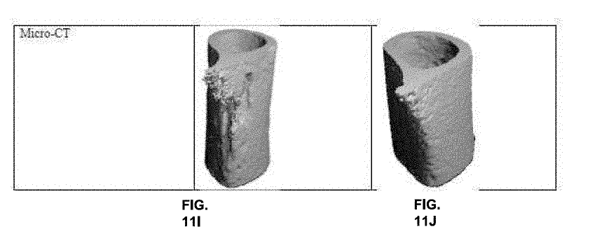

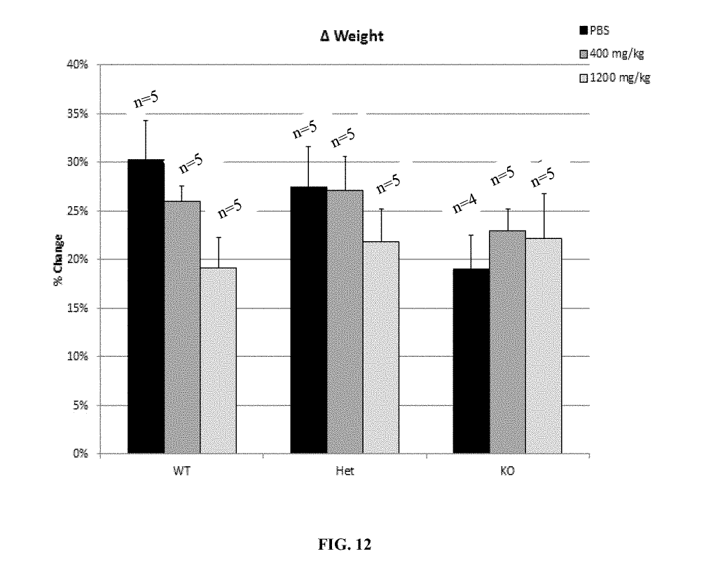

The same positive relationship between BMD and Miglustat was found in the TNSALP-null mice. Knockout mice administered PBS had a lower average mean/density of TV (apparent) (886.34 mg HA/ccm, n=2) than those injected with 400 mg/kg of Miglustat (968.03 mg HA/ccm, n=2) (Tables 16 & 18). Knockout mice bones were considerably more fragile and difficult to dissect. Homozygous microCT scans show clear evidence of improper mineralization at the diaphysis (FIGS. 10 & 11). MicroCT analysis of cortical bone also showed drastically thinner bones, in comparison to wild-type and heterozygous mice bones (data not shown). The PBS-treated knockout mice had the lowest average BMD of all eight treatment groups (Table 19 & FIG. 3).

However, the wild-type mice were an exception to this trend. The average mean/density of TV (apparent) was greater for wild-types administered 400 mg/kg (996.41±2.60 mg HA/ccm, n=5) than those that did not receive Miglustat (980.08±8.58 mg HA/ccm, n=4), but there was no change between the mice injected with 1200 mg/kg of Miglustat (974.48±3.63 mg HA/ccm, n=4) and the PBS wild-type treatment group (Tables 4, 6, & 8).

After one month of treatment, mice injected with Miglustat had higher average BMDs in comparison to the groups receiving PBS across all genotypes. The exception to this trend was one of the control groups (wild-type treatment group) that was injected with 1200 mg/kg of Miglustat. The general relationship demonstrates an association between increased Miglustat dosage and increased BMD.

Although the differences in average mean/density of TV (apparent) appeared to be small, they could still be considered substantial. The fact that murine HPP was observed in this study could serve as an explanation for the slight changes in BMD. In addition, the one month time frame may not have been sufficient to see more drastic changes, especially within cortical bone as it is not located near the growth plates. Moreover, because of their normal ALP activity levels and lack of Miglustat in injections, the average BMD of the wild-types on the PBS treatment served as a desired norm. Data showed that heterozygous mice were still able to match or even surpass this norm with high doses of Miglustat. In addition, the homozygous mice administered the 400 mg/kg dosage of Miglustat also exhibited a substantial difference in BMD between the PBS knockouts and the PBS wild-types with Miglustat treatment.

Because Miglustat affected the control group, the treatment most preferably should be prescribed for individuals with low BMD because of the potentially adverse effects of abnormally high BMD. The differences in BMD due to the varying doses of Miglustat could also indicate the use of Miglustat to treat a range of HPP symptoms. While severe forms of HPP such as infantile could be administered higher doses of Miglustat, more moderate cases could be administered lower doses to avoid resultant BMDs following administration that could be detrimental otherwise.

Not only did mice with abnormally low TNSALP activity levels have increases in BMD following Miglustat treatment, but Miglustat increased wild-type BMD for the 400 mg/kg group as well. This indicates that the mechanism behind Miglustat is not specific to the disease, but rather affects the bone mineralization process in general. Miglustat must play a role in bone mineralization independent of its already established ability to inhibit glycosphingolipid biosynthesis. Low BMD presents in patients of many bone diseases (e.g., osteopenia, osteoporosis, osteomalacia, and osteoarthritis). Therefore, Miglustat treatment could be expanded to reduce the effects of these bone diseases.

### Example 2

TNSALP-null (Akp2^{-/-}) mice served as an in vivo model of bone disease to analyze the effects of the iminosugar, Miglustat.

Mice were obtained, bred and genotyped as described above. Five mice of each genotype (Wild-type (WT), heterozygous (Het), and homozygous (KO)) were included in each treatment group (i.p. administration of PBS, 400 mg/kg Miglustat, and 1200 mg/kg Miglustat as described above). A total of 44 mice were analyzed (1 Akp2^{-/-} homozygous mouse was omitted in the PBS group). Mice were scanned using microCT and analyzed as described above. Cortical bone in the middle 20% of bone length from trochanter to distal condyles in the femur was scanned and cortical thickness and bone mineral density were obtained. Cortical thickness (Ct.Th, μm) was used as an indication of average thickness of cortical bone. Trabecular bone of the final 625 μm before the femoral growth plate was scanned.

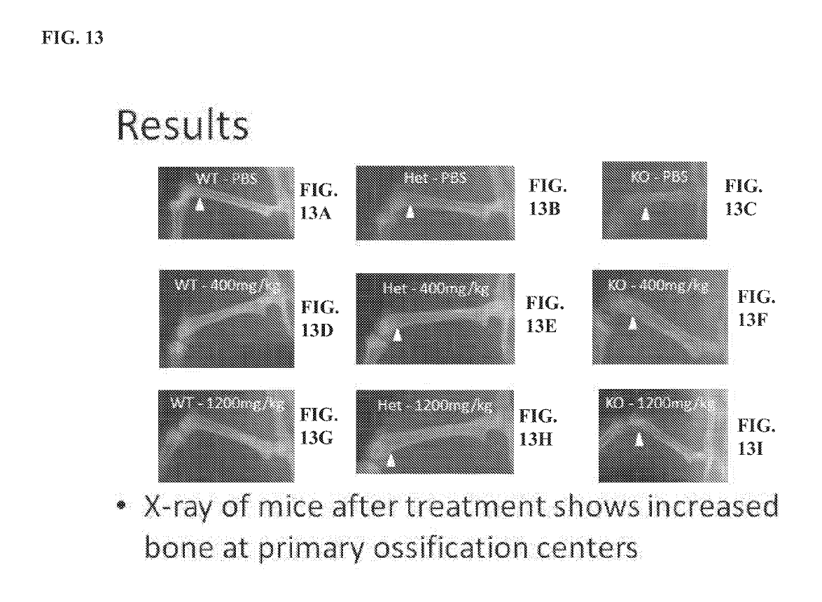

As depicted in FIG. 12, high doses of Miglustat impeded growth in mice as measured by percent change in weight. This was particularly notable in Wild-type (WT) and Heterozyous (Het) mice. As shown in the X-Rays in FIGS. 13A-13I, administration of Miglustat increased bone at primary ossification centers (arrowheads).

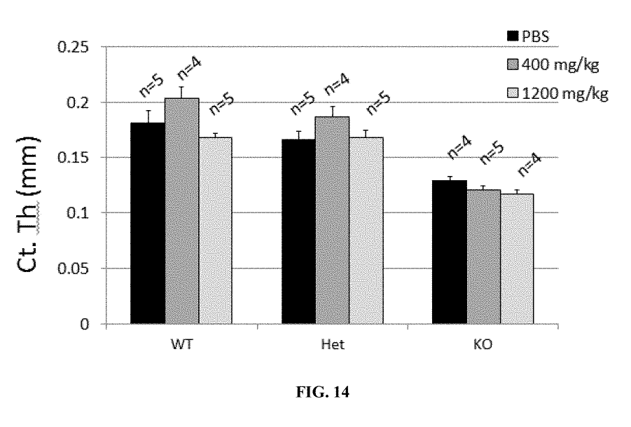

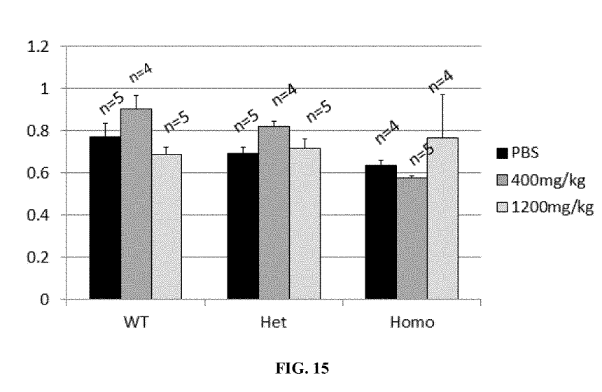

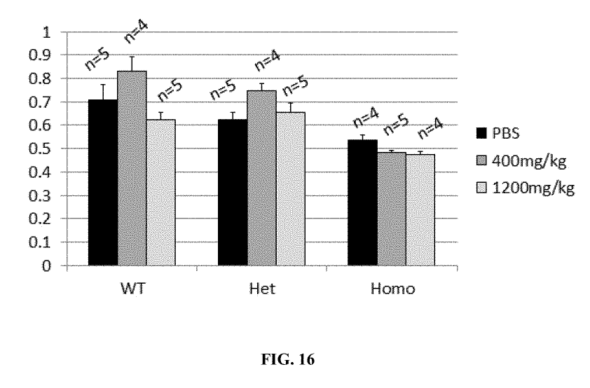

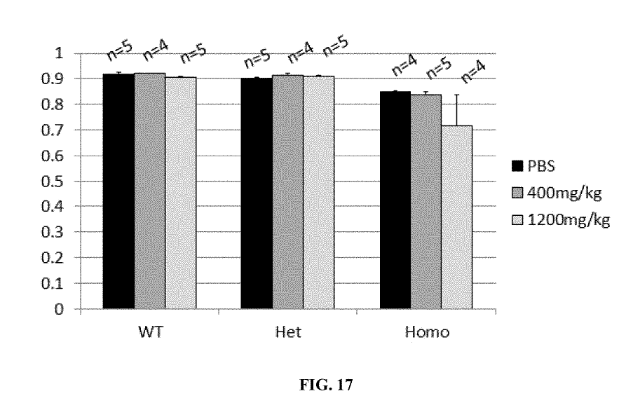

As depicted in FIG. 14, Miglustat improved cortical thickness in heterozygous mice at 400 mg/kg. As depicted in FIG. 15, there was a slight increase in total cross sectional area inside the periosteal envelope in Heterozygotes treated with 400 mg/kg Miglustat. Cortical bone area (Ct.Ar, mm²) was determined by dividing cortical volume by the number of slices X slice thickness. As depicted in FIG. 16, cortical bone area increased in Wild-type (WT) and Heterozygotes (Het) administered 400 mg/kg Miglustat. As depicted in FIG. 17, administration of Miglustat did not have an effect on normalized bone area-cortical bone volume fraction in Wild-type and Heterozygotes. In Homozygotes administered 1,200 mg/kg normalized bone area-cortical bone volume fraction decreased.

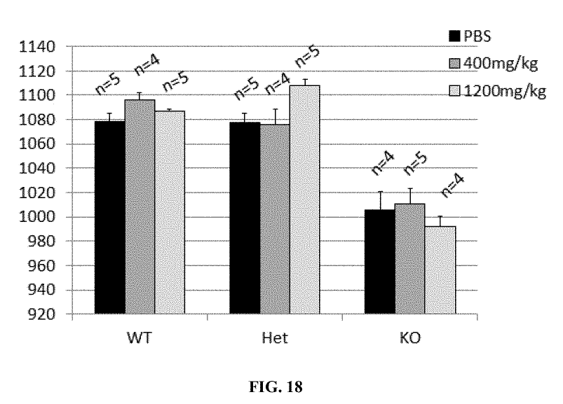

Tissue mineral density was calculated from the average attenuation value of the bone only and did not include attenuation values from non-bone voxels (=discrete unit of the scan volume that results from the tomographic reconstruction), as was done for BMD. The attenuation depended on physical density and electron density of bone. As depicted in FIG. 18, high doses of Miglustat improved tissue mineral density in Heterozygotes.

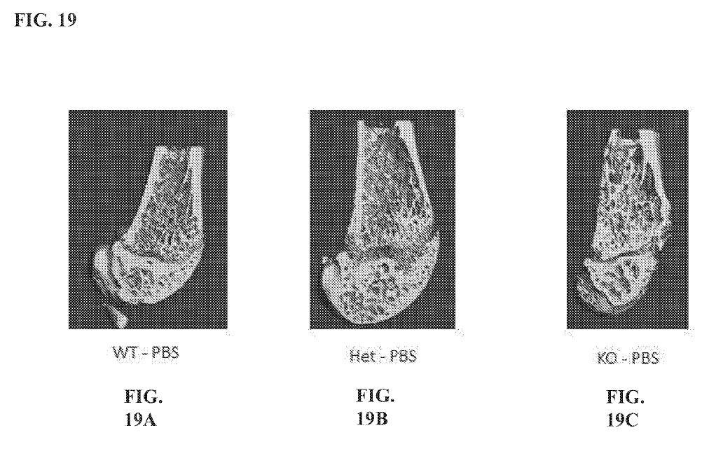

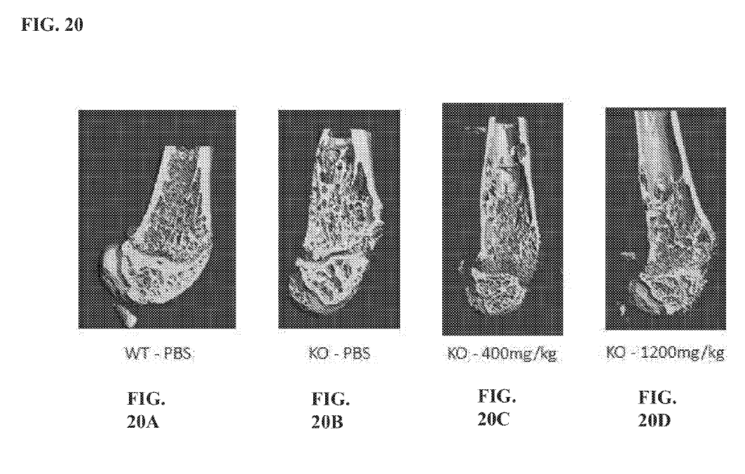

As depicted in FIGS. 19A-19C, Homozygotes (Akp−/−) had visually different trabeculae. Trabeculae were larger and fewer in number with more space between trabeculae (compare WT-PBS in FIG. 19A and Het-PBS in FIG. 19B with KO-PBS in FIG. 19C). As depicted in FIGS. 20A-20D, treatment with Miglustat appeared to restore the appearance of narrow trabeculae (compare WT-PBS in FIG. 20A and KO-PBS in FIG. 20B with KO-400 mg/kg in FIG. 20C and KO-1200 mg/kg in FIG. 20D).

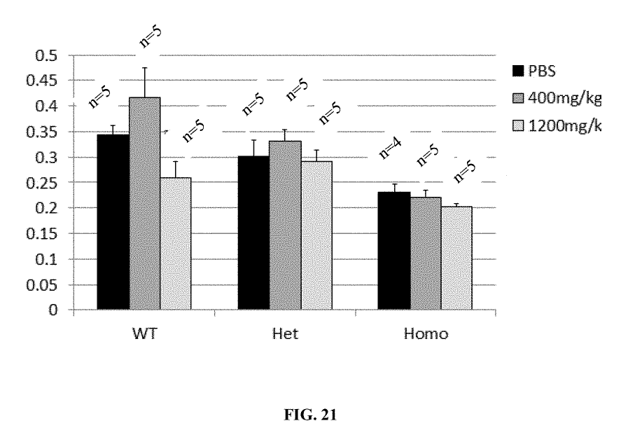

As depicted in FIG. 21, overall bone volume was not significantly increased after treatment. Bone volume fraction is the ratio of the segmented bone volume to the total volume of the region of interest.

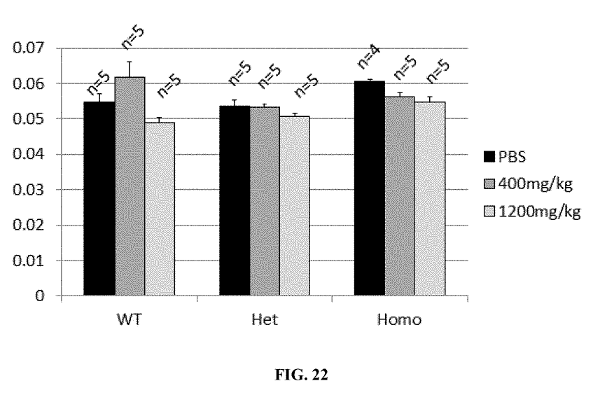

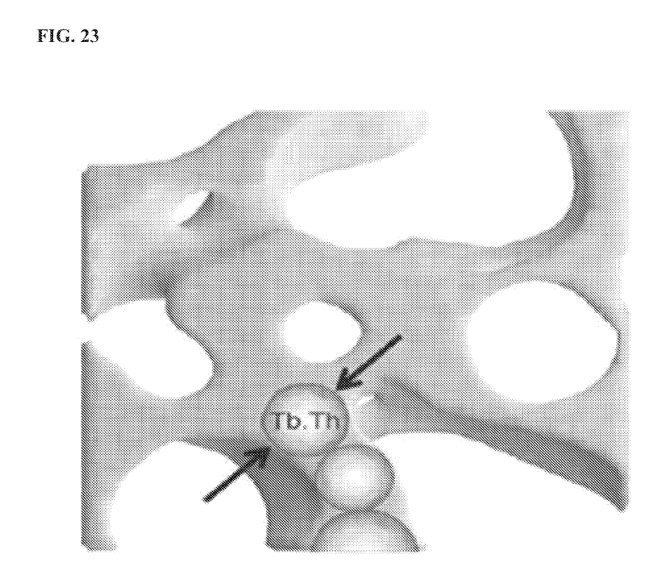

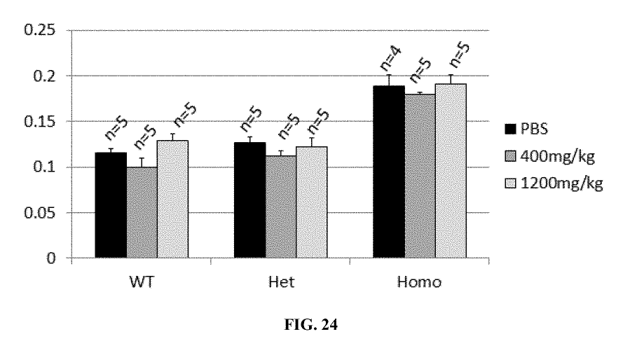

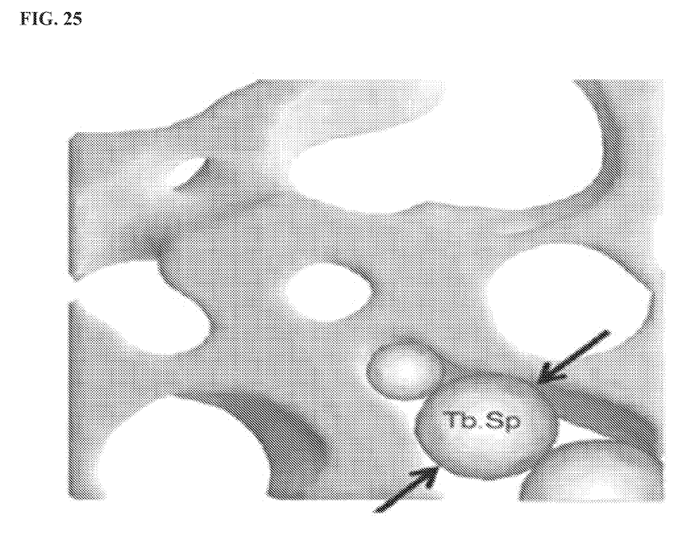

As depicted in FIG. 22, restoration of narrow trabeculae resulted in lower trabecular thickness in homozygotes. Trabecular thickness was measured as illustrated in FIG. 23. As depicted in FIG. 24, treatment resulted in a decrease in trabecular separation. Trabecular separation is the mean distance between trabeculae assessed using direct 3D methods as illustrated in FIG. 25.

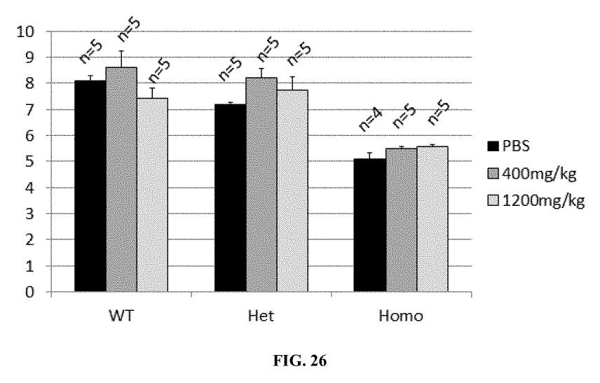

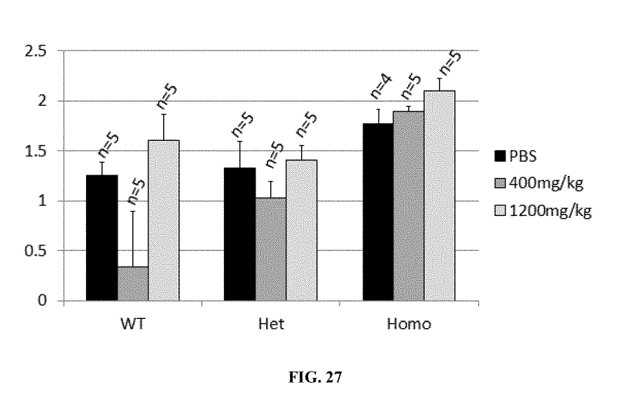

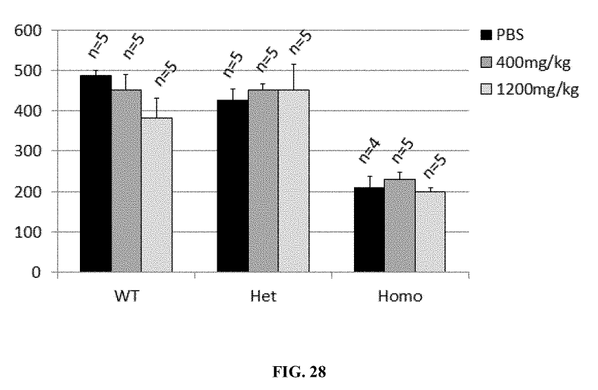

As depicted in FIG. 26, the trabecular number changed with Miglustat in both heterozygotes and homozygotes treated with Miglustat. As depicted in FIG. 27, structure model index (SMI) increased in homozygous AKP mice treated with Miglustat. This indicates that the trabecular bone structure changed to a more rod-like structure. SMI represents an indicator of the structure of the trabeculae. SMI will be zero for parallel plates and 3 for cylindrical rods.

As depicted in FIG. 28, connectivity density did not change in heterozygotes or homozygotes administered Miglustat. Connectivity density is a measure of the degree of connectivity of trabeculae normalized by total volume.

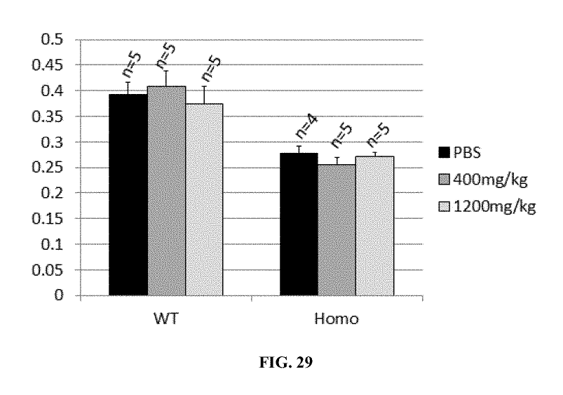

In addition to microCT analysis of femurs, microCT analysis was performed on trabecular bone in the lumbar vertebrae. As depicted in FIG. 29, overall bone volume was not significantly increased in homozygotes following treatment. Bone volume fraction of vertebrae is the ratio of the segmented bone volume to the total volume of the region of interest.

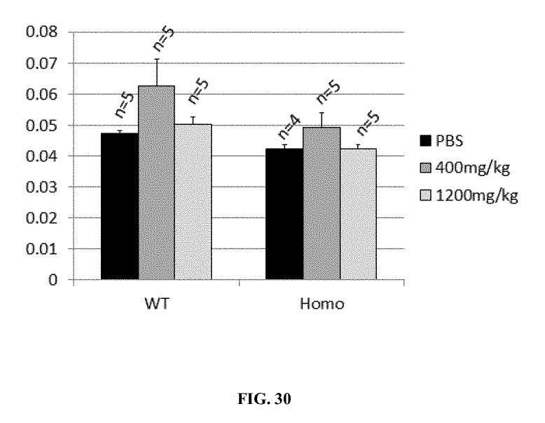

As depicted in FIG. 30, trabecular thickness (Tb.Th, mm) increased at 400 mg/kg of Miglustat. The profile of trabecular thickness in spine without treatment is different than femoral trabecular thickness. Trabecular thickness is the mean thickness or trabeculae assessed using direct 3D methods described above.

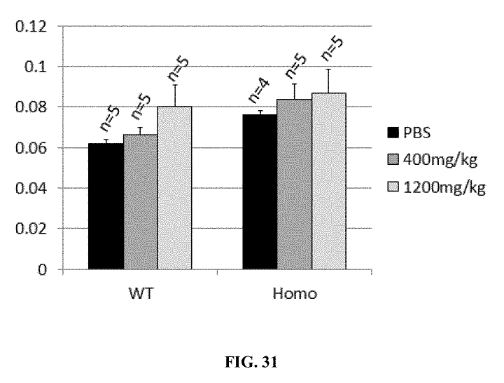

As depicted in FIG. 31, trabecular separation increased proportional to dose of Miglustat. The profile of trabecular separation in spine is different than in femoral. Trabecular separation is the mean distance between trabeculae assessed using direct 3D methods described above.

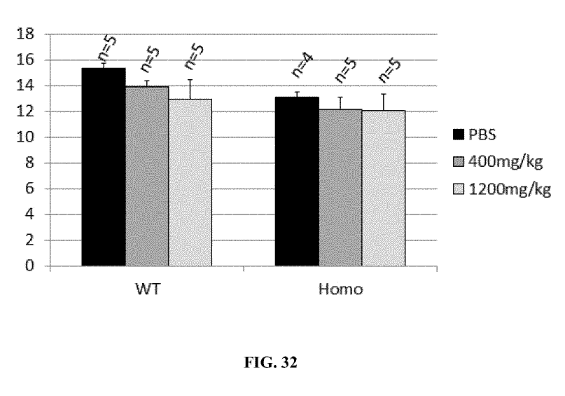

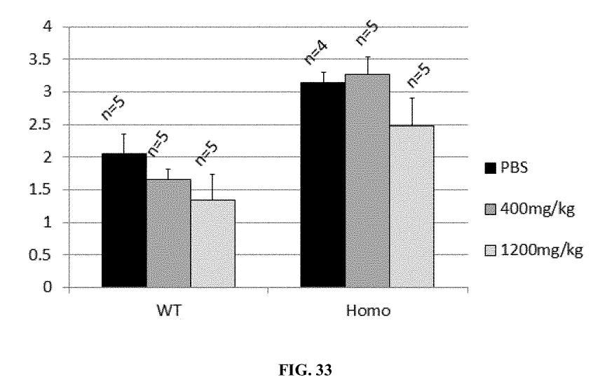

As depicted in FIG. 32, the trabecular number did not change significantly with Miglustat. The profile and baseline number of trabeculae in spine was different than in femoral. Trabecular number is the measure of the average number of trabeculae per unit length.

As depicted in FIG. 33, structure model index decreased in homozygote mice treated with Miglustat. This indicates that the trabecular bone structure changed from a rod-like structure to a plate structure which resembles the WT. As discussed previously, SMI is an indicator of the structure of the trabeculae. SMI will be zero for parallel plates and 3 for cylindrical rods.

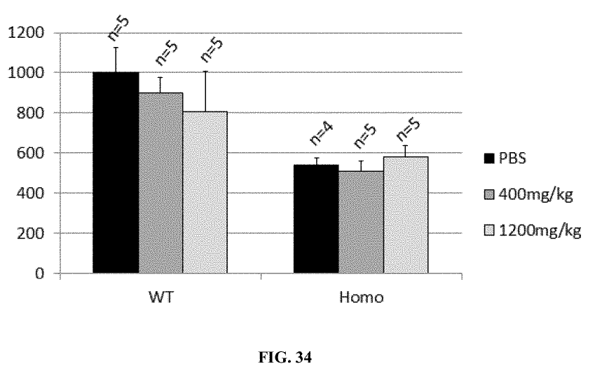

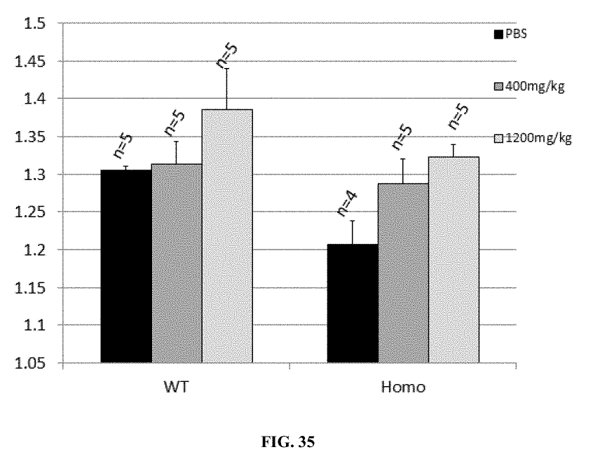

As depicted in FIG. 34, the connectivity density increased after treatment of homozygotes with high doses of Miglustat. As described above, connectivity density is a measure of the degree of connectivity of trabeculae normalized by total volume.

As depicted in FIG. 35, the degree of anisotropy increased with Miglustat meaning that treatment affected the orientation of trabeculae in the bone. Degree of anisotropy defines the direction and magnitude of the preferred orientation of trabeculae and uses the ratio between the maximum and minimum radii of the mean intercept length ellipsoid.

The results provided herein demonstrate the improvement in bone mineral density in an animal model of bone diseases by the administration of iminosugar. Notably, no toxicity was observed although 400 mg/kg/day (delivered intraperitoneally) corresponded to 13 times the recommended human oral dose based on body surface area and 1,200 mg/kg/day (delivered intraperitoneally) corresponded to 40 times the recommended human oral dose based on body surface area.

1. A method for treating a bone disease, the method comprising administering a composition comprising iminosugar to a subject in need thereof, wherein the subject has or is suspected of having a bone disease selected from the group consisting of osteopenia, osteoporosis, osteomalacia, and osteomalacia.

2. The method of claim 1, wherein the iminosugar is selected from the group consisting of multivalent iminosugars, sp2 iminosugars, iminosugar based galactosides, miglustat, eligustat, Bicyclic 1-Deoxygalactonojirimycin derivative and combinations thereof.

3. The method of claim 1, wherein the iminosugar in a dosage ranging from about 1 mg/kg to about 1,200 mg/kg.

4.-12. (canceled)

13. A method for treating hypophosphatasia, the method comprising administering a composition comprising iminosugar.

14. The method of claim 13, wherein the iminosugar is selected from the group consisting of multivalent iminosugars, sp2 iminosugars, iminosugar based galactosides, miglustat, eligustat, Bicyclic 1-Deoxygalactonojirimycin derivative and combinations thereof.

15. The method of claim 13, wherein the iminosugar in a dosage ranging from about 1 mg/kg to about 1,200 mg/kg.

17. A method for increasing bone mineral density, the method comprising administering a composition comprising iminosugar.

18. The method of claim 17, wherein the iminosugar is selected from the group consisting of multivalent iminosugars, sp2 iminosugars, iminosugar based galactosides, miglustat, eligustat, Bicyclic 1-Deoxygalactonojirimycin derivative and combinations thereof.

19. The method of claim 17, wherein the iminosugar in a dosage ranging from about 1 mg/kg to about 1,200 mg/kg.


SEQUENCE LISTING (3 sequences)

SEQ ID NO: 1 - DNA, 24 bp, Artificial Sequence
  Synthetic
agggggatgtgctgcaaggcgatt

SEQ ID NO: 2 - DNA, 24 bp, Artificial Sequence
  Synthetic
ctggcacaaaagagttggtaaggc

SEQ ID NO: 3 - DNA, 24 bp, Artificial Sequence
  Synthetic
gatcggaacgtcaattaacgtcaa